(19) United States
(12) Patent Application Publication
(10) Pub. No.: US 2004/0102808 A1
(43) Pub. Date: May 27, 2004

(54) Needle for retrieving a suture

(76) Inventors: Laveille Kao Voss, Belmont, CA (US); Erik Walberg, San Jose, CA (US)

Correspondence Address: BURNS, DOANE, SWECKER & MATHIS, L.L.P., P.O. Box 1404, Alexandria, VA, 22313-1404, US

(21) Appl. No.: 10/305,545

(22) Filed: Nov. 26, 2002

(51) Int. Cl. A61B 17/06

(52) U.S. Cl. 606/223; 606/138

(57) ABSTRACT

A needle having a shaft; a neck at the distal end of the shaft; and a tapered end at the distal end of the neck, the tapered end comprising at least three planar faces meeting at a central point, and an annular shoulder adjacent to the neck.

### TECHNICAL FIELD

The present invention relates to needles for suturing tissue, and to systems for retrieving and pulling sutures through tissues.

### BACKGROUND OF THE INVENTION

Surgical needles have long been used to advance sutures through patient tissues. In addition, however, surgical needles may be used to retrieve sutures. For example, the suture may be positioned on one side of a tissue and the needle may be positioned on the other side of the tissue. The needle is then advanced through the tissue to “snare” the suture. The needle is the pulled back through the tissue, pulling the suture therethrough. Although such suture retrieval systems may operate by snaring a central loop of suture, suture retrieval systems may also grab onto an end of the suture. For example, the end of the suture may be supported in a cuff that is built to engage the needle. Thus, the needle is advanced such that its end locks into the cuff. As the needle is withdrawn, it pulls the cuff which in turn pulls the suture therewith. An example of such a system is found in U.S. Pat. No. 6,136,010.

Problems common to all surgical or percutaneous needle device systems is that needles may break, or be deflected. This is especially true when the needles are advanced through scarred or calcified tissues. In device systems, where needles are deployed to hit a particular target, the ability of the needle to penetrate tough and calcified tissue is critical. Moreover, when space is a constraint, simply increasing the needle diameter to improve penetration is not feasible.

### SUMMARY OF THE INVENTION

The present invention provides a needle which is ideally suited to be advanced through a tissue mass, engage with a cuff that is holding the end of a suture, and retrieve the suture (i.e.: pull the suture back through the tissue) when the needle is withdrawn.

In preferred embodiments, the present needle includes a shaft; a neck at the distal end of the shaft; and a tapered end tip at the distal end of the neck. The tip comprises at least three planar faces. Preferably, these three (or more) planar faces intersect at a central point. Most preferably, this central point is co-linear with the central longitudinal axis of the shaft. Additionally, the proximal portion of the tip (i.e.: the portion adjacent to the neck) has a portion which is cylindrically shaped so as to form an annular shoulder.

In preferred embodiments, the at least three planar faces is exactly three faces, however, embodiments with four, five or even more faces are also contemplated within the scope of the present invention.

An advantage of the present invention is that the intersections of the planar faces form sharp cutting edges. These sharp cutting edges improve the needles' ability to penetrate tissue. Specifically, the sharp cutting edges decrease the amount of force required to advance the needle through body tissues. This is especially advantageous when pushing the needle through scarred or calcified tissues.

In preferred aspects, the neck is a symmetrically narrowed portion of the shaft, a large portion of which progressively narrows in its distal direction. Most preferably, the annular shoulder comprises a face that is generally perpendicular to the central longitudinal axis of the shaft. Therefore, the present invention can be inserted into (and engage or interlock with) a suture cuff such that a suture held therein can be pulled when the needle is withdrawn.

A further advantage of the present invention is that it improves cuff pickup ability by improved penetration of difficult tissue to the target cuff.

### BRIEF DESCRIPTION OF THE DRAWINGS

FIG. 1A is a side elevation view of a preferred embodiment the present needle having three planar faces meeting at a central location at its distal end.

FIG. 1B is a top view corresponding to FIG. 1A.

FIG. 1C is a bottom view corresponding to FIG. 1A.

FIG. 2 is an end view of the needle of FIG. 1.

FIG. 3 is an end view of a preferred embodiment the present needle having four planar faces meeting at a central location at its distal end.

FIG. 4 is an end view of a preferred embodiment the present needle having five planar faces meeting at a central location at its distal end.

FIG. 5 is a side elevation view of the needle of FIG. 1 positioned to be received into a suture cuff.

FIG. 6 is a sectional side elevation view corresponding to FIG. 5.

FIG. 7 is a view similar to FIG. 6, but with the needle advanced into the cuff such that tabs on the cuff lock the tip of the needle therein.

FIG. 8 is an end view of the cuff taken along line 8-8 in FIG. 6, showing the inwardly protruding tabs of the cuff.

### DETAILED DESCRIPTION OF THE DRAWINGS

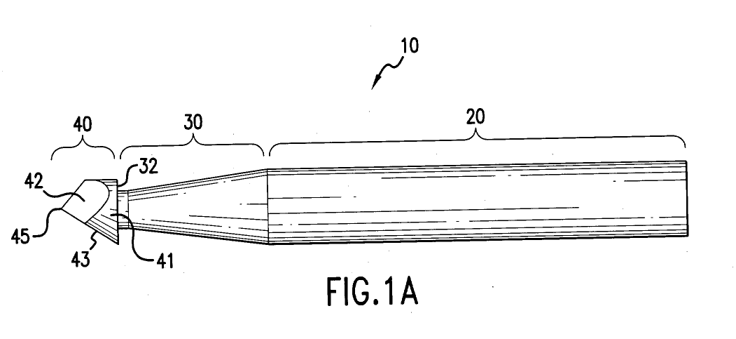

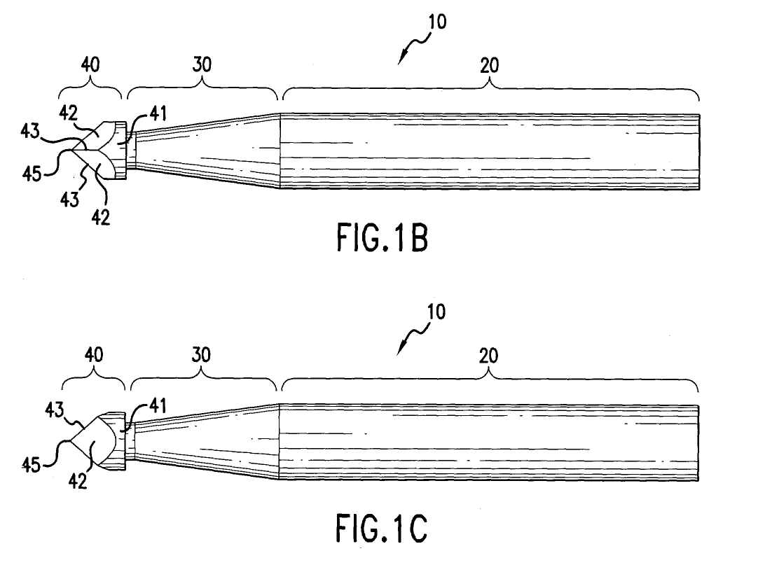

Referring to FIGS. 1A, 1B and 1C (hereafter, “FIG. 1”), the present invention provides a needle 10 having a shaft 20, a neck 30 and a tapered end tip 40. More specifically, the present invention provides a needle 10 having a shaft 20, a neck 30 at a distal end of shaft 20; and a tip 40 at a distal end of neck 30, tip 40 including at least three planar faces 42 and an annular shoulder 32 adjacent to neck 30.

The diameter of shaft 20 of needle 10 is preferably less than 30 mils. More preferably, diameter of shaft 20 of needle is from about 15 to 25 mils. Most preferably, the diameter of shaft 20 of needle 10 is about 20 mils.

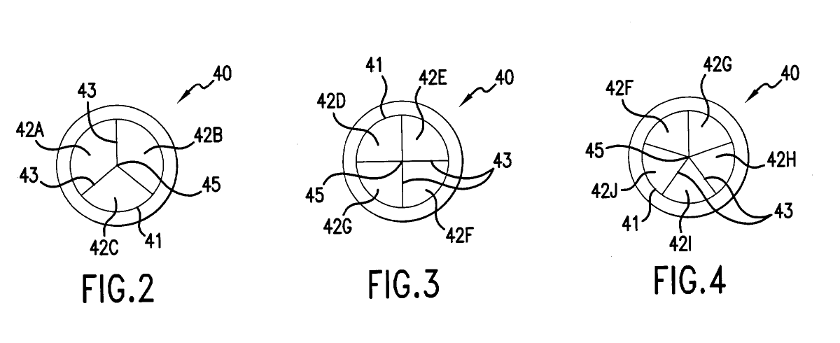

As seen in FIGS. 2, 3 and 4, tip 40 includes a plurality of planar faces 42 which together meet at a central point 45. For example, FIG. 2 shows a most preferred embodiment of the invention having three planar faces 42A, 42B and 42C which together meet at central point 45. Sharpened cutting edges 43 are formed at the intersections of planar faces 42A, 42B and 42C, as shown. Sharpened cutting edges 43 assist needle 10 in cutting through the patient's tissue as the needle is advanced, rather than simply dissecting tissue as in the case of a conically-shaped needle tip.

The dimensions of the needle are preferably selected such that the cutting edges are at angles that provide adequate tissue penetration, and such that the tip is sized to fit into, and engage, a cuff. Preferably, the diameter of annular shoulder 32 is from about 14 to 17 mils, and most preferably about 15.6 mils. Preferably, the length of tip 40 is from about 12 to about 18 mils, and most preferably about 15 mils. Preferably, the angle between any planar face 42 and a central longitudinal axis of the needle is between about 25 and about 35 degrees. Most preferably, the angle between any planar face 42 and a central longitudinal axis of the needle is about 31 degrees. Preferably, the angle between any cutting edge 43 and a central longitudinal axis of the needle is between about 40 to about 60 degrees. Most preferably, the angle between any cutting edge 43 and a central longitudinal axis of the needle is about 50 degrees.

It is to be understood that the present invention is not limited to embodiments having three planar faces 42. Rather, embodiments having more than three planar faces 42 are also contemplated, as illustrated in FIGS. 3 and 4 described below.

FIG. 3 shows an alternate embodiment of the invention having four planar faces 42D, 42E, 42F and 42G which together meet at central point 45. Sharpened cutting edges 43 are formed at the intersections of planar faces 42D, 42E, 42F and 42G, as shown.

FIG. 4 shows an alternate embodiment of the invention having four planar faces 42H, 42I, 42J, 42K and 42L which together meet at central point 45. Sharpened cutting edges 43 are formed at the intersections of planar faces 42H, 42I, 42J, 42K and 42L, as shown.

In various preferred embodiments, central point 45 may preferably be disposed at a location which is co-linear with the central longitudinal axis of shaft 20, as shown. An advantage of having central point 45 co-linear with the central longitudinal axis of shaft 20 is that it may be useful in accurately targeting the suture cuff.

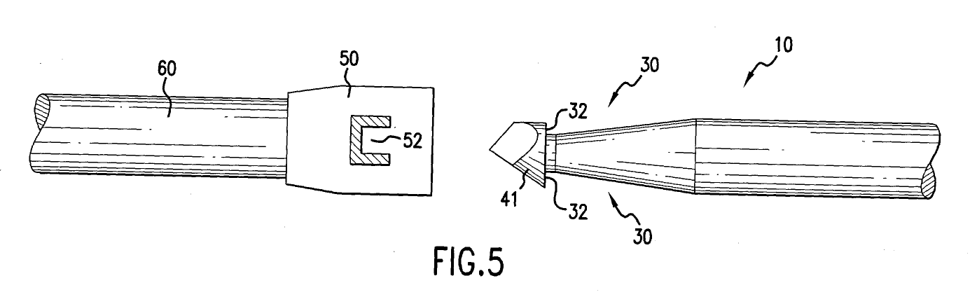

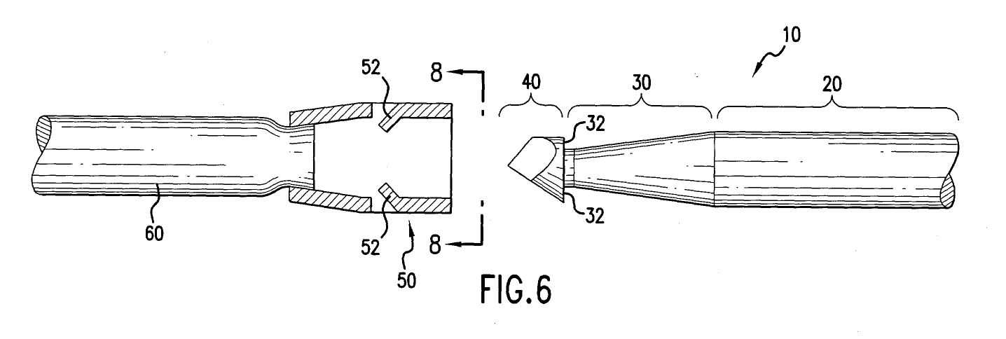

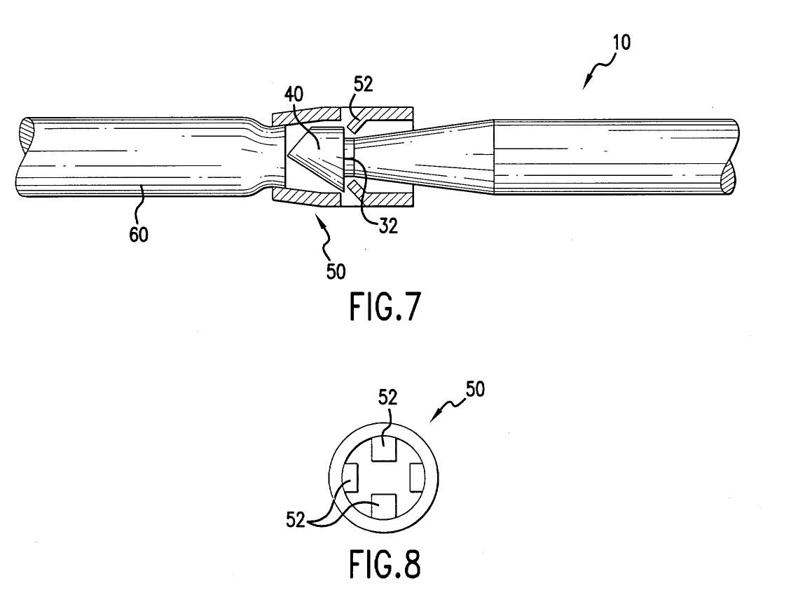

As shown in FIGS. 5, 6 and 7, the present invention further provides a system in which a needle 10 is ideally suited to interlock with a suture cuff 50 (which in turn holds an end of a suture strand 60 therein). Specifically, cuff 50 is crimped onto an end of suture 60, as shown. Cuff 50 includes a plurality of bent-in tabs 52. Tabs 52 are dimensioned such that they fit securely into neck 30 when needle 10 is advanced therein, as shown in FIG. 7. Thus, to ensure a secure fit between tabs 52 and neck 30, neck 30 preferably narrows (tapers) in the distal direction. Tip 40 may also preferably comprise a face 32 which is perpendicular to a central longitudinal axis through shaft 20 of needle 10. Face 32 forms an annular shoulder that provides resistance against tabs 52 such that the suture cuff 50 can not be removed from needle 10 once needle 10 is inserted therein. As can be seen, the annular shoulder formed by face 32 is formed by the proximal portion 41 of tip 40 (i.e.: the portion adjacent to neck 30) being cylindrical in shape.

In preferred embodiments, either or both of the needle and the suture cuff may be made from stainless steel. However, the present invention is not so limited. Other steels, and other materials, for example, Nitinol™ may be used instead.

1. A needle comprising:
a shaft;
a neck at a distal end of the shaft; and
a tip at a distal end of the neck, the tip comprising an annular shoulder adjacent to the neck and at least three planar faces.

2. The needle of claim 1, wherein the at least three planar faces intersect to form at least three cutting edges.

3. The needle of claim 1, wherein the at least three planar faces intersect at a central point that is co-linear with a central longitudinal axis of the shaft.

4. The needle of claim 1, wherein the annular shoulder is formed by a cylindrical portion of the tip.

5. The needle of claim 1, wherein at least a portion of the neck is tapered inward such that the neck narrows in a distal direction.

6. The needle of claim 1, wherein at least a portion of the neck is cylindrical.

7. The needle of claim 1, wherein the annular shoulder comprises a surface having a circular circumference, the surface being generally perpendicular to the central longitudinal axis of the shaft.

8. The needle of claim 1, wherein the tip comprises exactly three planar faces.

9. The needle of claim 1, wherein the tip comprises more than three planar faces.

10. The needle of claim 1, wherein the diameter of the shaft of the needle is less than about 30 mils.

11. The needle of claim 1, wherein the diameter of the shaft of the needle is from about 15 to about 25 mils.

12. The needle of claim 1, wherein the diameter of the annular shoulder is 14 to about 17 mils, and the length of the tip is from about 12 to about 18 mils.

13. The needle of claim 1, wherein the angle between at least one of the three planar faces and a central longitudinal axis of the needle is between about 25 and about 35 degrees.

14. The needle of claim 2, wherein the angle between at least one of the cutting edges and a central longitudinal axis of the needle is between about 40 to about 60 degrees.

15. A needle comprising:
a shaft;
a neck at a distal end of the shaft; and
a tip at a distal end of the neck, the tip comprising three planar faces intersecting at a distal end of the tip, and an annular shoulder formed by a cylindrical portion of the tip adjacent to the neck, wherein the angle between at least one of the three planar faces and a central longitudinal axis of the needle is between about 25 and about 35 degrees.

16. The needle of claim 15, wherein at least a portion of the neck is tapered inward such that the neck narrows in a distal direction.

17. The needle of claim 15, wherein at least a portion of the neck is cylindrical.

18. The needle of claim 15, wherein the annular shoulder comprises a surface having a circular circumference, the surface being generally perpendicular to the central longitudinal axis of the shaft.

19. The needle of claim 15, wherein the diameter of the annular shoulder is from 14 to about 17 mils, and the length of the tip is about 12 to about 18 mils.

20. The needle of claim 15, wherein the angle between at least one of the three planar faces and a central longitudinal axis of the needle is between about 25 and about 35 degrees.

21. The needle of claim 15, wherein the at least three planar faces intersect to form at least three cutting edges, and wherein the angle between at least one of the cutting edges and a central longitudinal axis of the needle is between about 40 to about 60 degrees.

22. A system for retrieving a suture, comprising:
a needle comprising:
a shaft;
a neck at the distal end of the shaft; and
a tip at the distal end of the neck, the tip comprising an annular shoulder adjacent to the neck and at least three planar faces opposite to the neck;
a suture; and
a cuff at an end of the suture, the cuff comprising a cylindrical wall defining at least one inwardly projecting tab, the at least one tab positioned to engage the annular shoulder of the tip of the needle when the tip of the needle is inserted into the cuff.

23. The needle of claim 22, wherein at least a portion of the neck is tapered inward such that the neck narrows in a distal direction.

24. The needle of claim 22, wherein at least a portion of the neck is cylindrical.

25. The needle of claim 22, wherein the annular shoulder comprises a surface having a circular circumference, the surface being generally perpendicular to the central longitudinal axis of the shaft.

26. The needle of claim 22, wherein the diameter of the annular shoulder is from 14 to about 17 mils, and the length of the tip of the is about 12 to about 18 mils.

27. The needle of claim 22, wherein the angle between at least one of the three planar faces and a central longitudinal axis of the needle is between about 25 and about 35 degrees.

28. The needle of claim 22, wherein the at least three planar faces intersect to form at least three cutting edges, and wherein the angle between at least one of the cutting edges and a central longitudinal axis of the needle is between about 40 to about 60 degrees.
